(19) United States
(12) Patent Application Publication
(10) Pub. No.: US 2002/0155086 A1
(43) Pub. Date: Oct. 24, 2002

(54) Hair and scalp treatment composition

(76) Inventors: Peter C. Verdun, Los Angeles, CA (US); Agnes Verdun, Los Angeles, CA (US)

Correspondence Address: ROBERT J. SCHAAP, Suite 188, 21241 Ventura Boulevard, Woodland Hills, CA, 91364, US

(21) Appl. No.: 09/781,659

(22) Filed: Feb. 9, 2001

(51) Int. Cl. A61K 7/06

(52) U.S. Cl. 424/74

(57) ABSTRACT

A hair and scalp treatment composition for generally improving the health of hair and scalp of an individual and also improving the manageability of the hair. The composition is primarily based on the use of herbal components and does not employ any strong chemical reagents. The composition will also aid in maintaining normal hair color tending to obviate some of the aging conditions and will even aid in some hair growth. The composition is generally provided in the form of a wax or a gel and relies upon various major components, such as citronella and willow and bergamot, potentially along with certain vitamins which are initially mixed in selected vegetable oil where elements of these components are entrapped in the oil and the oil is then mixed in a wax or gel.

### BACKGROUND OF THE INVENTION

1. Field of the Invention

This invention relates in general to certain new and useful improvements in hair and scalp treatment compositions and, more particularly, to compositions which improve the health of the hair and scalp without using any strong chemical reagents or agents which rely upon a strong oxidation or reduction reaction with the hair or scalp and thereby avoids any adverse reactions with the user.

2. Brief Description of the Related Art

The prior art is replete with hair care products and where the producers of these products boast various claims with regard to color, aging, health and the like. In many cases, the claims have dubious authenticity and, in most cases, the products rely upon strong oxidizing or reducing agents to achieve a certain result. Thus, some hair care products are designed to overcome the problems of hair graying and inevitably rely upon strong oxidizing or strong reducing agents. Otherwise, they rely upon harsh dyes which frequently have adverse reactions with the scalp or other portions of the skin.

Some of the commercially available hair care products include braid sprays, vitalizers, detangling compositions, conditioners, gels for grooming, hair straighteners, hair dyes, and hair coloring agents. Although each of these various prior art compositions may be tailored to achieve one of more specific results, they all nevertheless rely upon harsh agents which have adverse effects to the hair and the scalp.

There are also numerous hair care products, such as those containing the chemical reagent Monoxidyl for allegedly improving hair growth. These products similarly suffer from the use of strong reagents which, again, can have adverse reactions with an individual.

Frequently, with many of these commercially available hair care products, the adverse conditions which result are frequently long term, in that the user does not experience the adversity of the product for many years and, perhaps, even for years after use of the prior art composition has ceased. These adverse conditions may take the form of hair fraying, lack of manageability, premature loss of hair, and the like. Equally as often, there is little or nothing medical science can do in order to abate or otherwise overcome the adversity which results from the earlier use of these harsh compositions.

Scalp dryness and dandruff are other problems which are associated with social sigmas and, here again, there are a plethora of commercially available products with attendant claims to overcome scalp dryness and dandruff. In this case, many of the prior art compositions which rely upon selenium and selenium oxide are effective for their intended purpose. However, they are also harsh acting chemicals which again create the various adverse conditions mentioned above.

There are also numerous hair care products which boast of the abilities to improve the luster or shine of a person's hair and the manageability of the hair. Usually, these products are incorporated in rinses or in shampoos, which again have strong acting chemical reactants. As a result, and although they may have the capability of providing the boasted result, they frequently also result in damage to the hair, as well as to give rise to the other adverse conditions mentioned above.

There has been a need for a composition which can provide various hair and scalp care treatments, but which does not result in the adverse effects of the type mentioned herein. There has also been a need for a health hair care product which can act as a revitalizer, a detangler, conditioner, a gel and a straightener, and which similarly does not present those mentioned adverse conditions.

### OBJECTS OF THE INVENTION

It is, therefore, one of the primary objects of the present invention to provide a primarily natural and healthy hair care product which provides capacities of revitalizing and conditioning the hair without the adversities associated with harsh chemical reacting products.

It is another object of the present invention to provide a hair care product of the type stated which is also capable of detangling and serving as a gel and a straightener, again without the associated disadvantages resulting from strong chemical acting agents.

It is a further object of the present invention to provide a hair care product of the type stated which is capable of being provided in various forms, including liquid form, gel form or wax paste form and which all have the same effectiveness.

It is an additional object of the present invention to provide a hair care product of the type stated which can be made of all natural ingredients and which still can be provided at a relatively low cost.

It is another salient object of the present invention to provide a method of producing a primarily natural hair care product without the adversities associated with strong chemical acting agents in those products.

With the above and other objects in view, our invention resides in the novel features of form, construction, arrangement and combination of the components in the composition and in the method of producing same as presently described and pointed out in the claims.

### BRIEF SUMMARY OF THE INVENTION

A hair and scalp treatment composition which primarily relies upon natural ingredients which present little or no adversity to the human body and largely rely upon the use of herbal ingredients.

Initially, herbal ingredients are mixed in one or more vegetable oils and allowed to sit for at least several days at room temperature. Thereafter, and when the active ingredients of the herbal components have been sufficiently absorbed (as determined by the color of the carrier oils), the oils are decanted from the otherwise dry ingredients. The dried herbal ingredients are then discarded and the oils are thereafter mixed in a gel or wax base. When mixing, the temperature of the oils is increased at very least to substantially facilitate the dissolving of the oils in the wax or gel base. Additional mixing may occur if required at that point.

One of the major ingredients in the composition which is formed is a hair growth stimulant, such as the natural herb bergamot. Other stimulants could be used for this purpose. A fungicide, again in a natural state, such as citronella, is another one of the major components in the composition of the present invention. In this respect, several natural fungicides are available for this purpose and include not only citronella, but a eucalyptus oil and several others, as hereinafter described.

A dry scalp and dandruff reducing agent, again in a natural state, is also one of the natural ingredients which may be used and include, for example, willow. The above ingredients are the main ingredients which are normally found in this type of composition. However, other components are frequently employed in the composition and include, for example, a hair manageability agent which may also provide a shine or brilliance to the hair. One of the natural ingredients which may be used for this purpose is the natural herb rosemary.

A natural coloring and straightening agent may also be optionally added to the composition. Again, this agent would be a natural one, such as the herb alkanet. The alkanet provides a natural type of color dye to the hair, although other dyes could be employed for this purpose.

It is preferable to almost always use vitamin E in the composition, although optionally vitamin A may be used in place of or in addition to the vitamin E. Vitamin E is effective in reducing the possibility of clogged pores. This is particularly important in the scalp region inasmuch as clogged pores frequently give rise to adverse scalp conditions and poor hair conditions.

A preservative, such as, for example, benzoin may also be introduced into the composition in order to increase shelf life of the composition. Again, many natural preservatives are available for this purpose and can be used in the composition.

This composition has been tested and found to be highly effective in achieving a stimulation of hair growth and improvement of hair condition and scalp condition. It is also effective in reducing the incidents of aging as, for example graying and the like. In addition, the composition operates as an effective conditioner to condition the scalp, again, with primarily natural ingredients.

It is important in connection with the present invention to insure that the composition is made essentially with all natural ingredients. Although it is possible to use one or more ingredients, such as stabilizing agents and the like, which are not necessarily natural, it is preferred to prepare the composition with primarily natural ingredients. In this respect, herbal ingredients are even more preferred. The compositions thus made in this manner provide the beneficial results, but are not accompanied by the harsh damage and injury which can result from the other commercially available hair and scalp treatment compositions.

The present invention fulfills these and other objects in the provision of a hair and scalp treatment composition which meets and fulfills all of the above-identified objects and others which have not been specifically enumerated. Various examples of this composition have been set forth and alternatives for the ingredients in the composition and the ranges of amounts have also been set forth. The details of the composition and the method of making same are more fully described in the following details description of the invention. However, it should be understood that this following description and the illustration of the method used to make the composition is only set forth for purposes of illustrating the general principles of the invention and are not to be taken in a limiting sense.

### BRIEF DESCRIPTION OF THE DRAWINGS

Having thus described the invention in general terms, reference will now be made to the accompanying drawing in which:

FIG. 1 is a schematic block diagram showing the steps and components involved in the method of making the composition of the present invention.

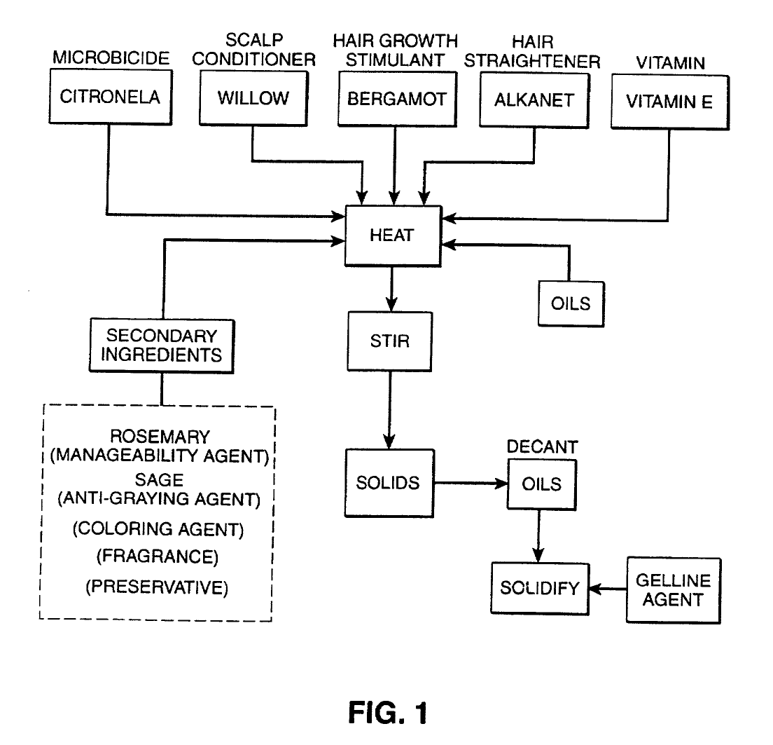

### DETAILED DESCRIPTION OF PREFERRED EMBODIMENTS

Referring now in more detail and by reference characters to the drawings, the hair and scalp treatment composition of the present invention is described primarily in one preferred embodiment with numerous equivalents thereof and the various possible embodiments of the invention which can be derived by using the equivalents for the ingredients in the preferred composition and the differing ranges thereof.

It is desired to produce a composition which is essentially an all natural composition and even one more preferably made entirely of herbs and natural ingredients. However, it is recognized that in some cases, other ingredients which are not necessarily herbal ingredients and which are even more so not natural ingredients, may also be used. However, in accordance with the present invention, it is preferable to keep the number of synthetic ingredients to a minimum, such that the composition is largely or primarily herbal or natural.

The composition of the present invention primarily starts with oils which are used as an initial liquid carrier. The oils are preferably natural oils, such as natural vegetable oils, although there could be mixtures of oils employed and which may contain some synthetic oils, as well. The preferred oils are soybean oil and olive oil. Both of these oils can be used in combination. The soybean oil predominates in amount of oil present constituting over 80% of the oil present by weight and the olive oil constitutes the significantly lesser percentage of oil, although these respective ranges can vary somewhat. When only one oil is used, it is preferred to use the soybean oil. Moreover, when both oils are used, it is preferable to use at very least 70% by weight of the soybean oil.

The citronella oil, sometimes referred to as cymbopogen oil, contains large amounts of citral and geraniol and which are naturally lemon-scented and rose-scented, respectively. The citronella is an aromatic herb which actually has cooling properties, in that it increase perspiration and relieves spasms. To this extent, it is effective for use as a scalp treatment. Moreover, the citronella oil is also known to be an effective bacterial agent in combating bacterial infections.

Citronella oil is also known for internal use in reducing digestive problems, particularly in children. It is also known for external use for treatment of ringworm, lice, athlete's foot and scabies. The oil has actually found use in herbal bath preparations and cosmetics. However, the citronella does find very effective use as a scalp and hair treatment.

The soybean oil has been known to be effective for reducing the incidents of clogged pores for many years. It is believed that the active ingredient in the soybean oil which is effective for this purpose is the lecithin. The olive oil has similarly been known to be useful for this purpose. However, it is possible to use other natural oils and, particularly, other natural vegetable oils as, for example, corn oil, almond oil, as well as oils of various other nuts.

Although it is possible to use animal fat oils, they are not preferred due to their high fat content. Nevertheless, they are natural to the extent that they are generated in animal bodies and are usually not as disadvantages as many of the lower alkene aromatic oils and synthetic oils.

As indicated previously, the major ingredients are added to the one or more oils in a mixture some of these other ingredients are normally in a dry state and still others are in liquid state. These ingredients are preferably, although not necessarily, all natural ingredients as, for example, herbal ingredients.

One of the main important ingredients added to the oil carrier is a microbicide for eliminating fungus or other microbial growth on the scalp of an individual. It is understood that microbial growth which can infest the scalp is often contributory to a large number of poor hair conditions and poor scalp conditions. Therefore, it is an important aspect of the present invention to eliminate these microbes which may have infested the scalp region.

One of the important microbicides which can be used is that of citronella. This ingredient is a natural insect repellant and is also highly effective in destroying fungus and precluding the growth of fungus on the scalp. As indicated previously, a eucalyptus oil can be used in place of the citronella oil or in addition to the citronella oil. Again, where a combination of the citronella oil and the eucalyptus oil is employed, the citronella oil would be used in as a substantial major portion of any such mixture. Thus, the citronella oil would be used in an amount of about at least 75% by weight and, even more preferably, an amount of about 85% by weight with respect to any other oil.

Any agent which is capable of operating as a microbicide can be used for this purpose. Thus, for example, iodine is also effective as a microbicide. However, iodine is not preferred inasmuch as it is not necessarily one of the natural herbal ingredients.

It is further desirable to use an anti-bacterial agent along with the microbicide. One highly effective anti-bacterial agent which is herbal in nature is alkanet sometimes known as Alkanna Tinctoria. Several species of the alkanets can be used. The alkanets are effective in that they actually function as a dye but more importantly operate as anti-bacterial herbs which promotes the healing and relieves itching of the scalp, particularly when the scalp may be injured by brushes or combs. The alkanets are also an effective astringent. Typically, alkanets have been used externally in the past for varicose and indolent ulcers, bed sores and itching rashes. Nevertheless, it has been found to be effective in the composition of the present invention.

Another ingredient which is important for use in the composition of the invention is a scalp conditioner, such as willow. The scalp conditioner preferably should act as a dandruff and flake control agent. It is know that the epidermal skin of the scalp will flake as a result of dryness and result in the condition known as dandruff. Although several selenium compounds are known as an effective scalp conditioning agent to control dandruff, they are not necessarily benign with respect to the condition of the scalp. It is therefore preferable to employ a herbal type ingredient.

The composition of the invention also includes a hair growth stimulant and, again, preferably a natural or herbal type hair growth stimulant. One of the most effective stimulants is that of bergamot, which contains a volatile oil comprising compounds relating to thymol and tannic acid. The use of bergamot has actually be found to be particularly good for depression as well as aiding the body in fighting of infections. Presently, it is used as a perfumery in cosmetics. Bergamot has also been known to be used for internal application to treat colic in babies and is externally used in douches and baths and for treatment of vaginal infections.

Willow, and preferably the bark of the will tree, is highly effective for this purpose. The willow not only conditions the scalp, but provides a natural oil to the scalp and reduces dandruff and scalp dryness. Other type herbal scalp conditioners may also be used include, for example.

The composition of the invention preferably includes a hair manageability agent which provides some luster or shine to the hair and also improves the texture and fullness of the hair. One of the preferred natural ingredients which can be used for this purpose is the herb, rosemary. This herb has been found to improve the sheen and texture of the hair. It is not fully understood if the rosemary or equivalent herbs, as hereinafter described, actually penetrate the hair or merely coat the hair. Nevertheless, rosemary and equivalent herbs have been found to be effective in operating as a hair manageability agent.

The rosemary is particularly effective as a hair manageability agent, since is also provides a sheen to the hair after use. In addition, the rosemary is known to literally penetrate the hair or otherwise coat the hair in order to provide more body to the hair, that is, to make it fuller in at least feel. The use of rosemary after rinsing of the hair also improves the texture and fullness of the hair.

Some of the other natural herbal ingredients which can be used for this purpose include that herb known as Irish moss. Although the odor from Irish moss is not as pleasant as that of rosemary, it is nevertheless an effective hair manageability agent. Other equivalent hair manageability agents which can also be used.

The composition of the invention also includes a mild hair coloring agent. One of the preferred natural hair coloring agents is that herb known as alkanet. In this case, alkanet also operates as a straightening agent to overcome curly hair.

The alkanets mentioned above are also highly effective in that they have already been used previously to make hair dyes with natural ingredients. The alkanets provide a slightly red to red-brown color to the hair, although other coloring agents could be used for this purpose. Some of the other natural hair coloring agents which may be used include, for example, some of the red dyes, such as the red dyes hereinafter described.

It is also possible to use hair coloring agents as, for example, several of the red dyes, such as red dye #2, red dye #5, possibly red dye #3, and the like. However, as indicated previously, it is preferable to use the natural ingredients, although some minor amounts of synthetic ingredients can be tolerated in the present invention. It is should also be recognized that in order to obtain colors other than red, other ingredients could be used.

It is, however, preferable in selecting a coloring agent to avoid the synthetic coloring agents and, particularly, the rather harsh acting oxidizing and reducing agents normally used for bleaching and dying hair.

Another one of the important ingredients used in the composition of the present invention is a vitamin, such as, and even preferably, vitamin E. Vitamin A also may be used in place of or in addition to the vitamin E, although the vitamin E has been found to provide the most beneficial results of all of the vitamins. Vitamin E operates in a manner similar to lecithin, in that it tends to eliminate clogged pores and actually operates to open clogged pores. Thus, vitamin E actually improves scalp condition.

Vitamin A can also be used in combination with vitamin E. Vitamin A has been found to have some benefit in healing qualities. It is recognized that when brushing hair and even combing hair with implements, such as the conventional brushes and combs, that some injury, even though slight, does result to the scalp. In this case, it has been found that the combination of vitamins E and A is beneficial in reducing the effects of those injuries and in healing those injuries.

Beta carotene has also been found to be effective as a substitute for vitamin A or vitamin E. Moreover, it can also be used in combination with vitamin A or vitamin E. When used in combination, however, it is preferable to include at least 50% of the vitamin E.

The composition of the invention may also use an anti-graying compound and which may be, for example, paba.

It is, however, possible to use other anti-graying compounds, such as, for example, equivalent compounds. Paba, for example, which is frequently used as an ingredient in suntan lotions. It is also possible to use other agents, such as panacinic acid. Some of the other anti-graying agents which may be used include the natural herb nettle and sage.

The herbs nettle or sage are both equally effective in the composition of the invention for serving as an anti-graying compound. The sage is an excellent ingredient inasmuch as it also helps to maintain the hair in a natural color. The use of sage has been well known at least by native Americans even prior to settling by Caucasians in order to prevent graying.

If desired, one can also add a fragrance to the composition and again preferably a natural fragrance ingredient, such as lavender. However, other fragrances can be used, including banana, strawberry, root beer, etc. Actually, each of these fragrances are naturally derived. However, other fragrances could be added if desired.

Inasmuch as some of the ingredients in the composition can be labile, particularly in the presence of water, it is frequently desirable to add a preservative. One of the highly effective preservatives is benzoic acid. Actually, benzoin gum in herbal form is also effective for this purpose and does not present the acidity as is presented by the benzoic acid. Benzoin, sometimes referred to as gum Benjamin, is also known to provide astringent and expectorant properties. Moreover, it is an effective herbal antiseptic. In Chinese medicine, the use of benzoin is regarded as a circulatory stimulant. To this extent, it is believed that the benzoin not only acts as a preservative, but it also is effective to improve circulation on the scalp of the user. Benzoin has been actually used as an anti-oxidant in cosmetics and a fixative in perfumes, creams and ointments. However, its ability to act as a circulatory stimulant is particularly effective as a hair and scalp treatment component in the present invention. Nevertheless, this is a preservative action, other well known preservatives may be used.

The main ingredients which are generally employed in the dry state and added to the aforesaid oils include the microbicide, such as citronella, the scalp conditioner, such as willow, the hair growth stimulant, such as bergamot, the coloring agent, such as alkanet and the vitamin, such a vitamin E. The most important of the aforesaid ingredients are possibly the hair strengthener, such as alkanet, the microbicide, such as citronella, the hair growth stimulant, such as bergamot, and the preservative, such as benzoin, and the latter of which actually functions more than a mere preservative, as aforesaid. One or more of these ingredients can be eliminated, however, they are preferred in the composition of the invention. In addition, it is also desirable to use the other ingredients mentioned above.

The ingredients which are added to the carrier oils, such as soybean and olive oil, are partially liquid, as described above, and include, for example, a vitamin E oil, bergamot in the form of an oil of bergamot, citronella in the form of an oil of citronella. The willow, such as white willow, the alkanet and benzoin gum are generally in a solid state. The benzoin gum is hereafter described, but for purposes of the invention, is usually introduced in this solid state.

In preparing the composition of the present invention, the aforesaid dry ingredients are added to the initial carrier oil or carrier oil mixture. This mixture is allowed to sit for at least several days at room temperature in order to allow the active ingredients of the dry components to be absorbed into the oil or oil mixture. It is possible to heat the oil with the solid components therein slightly to a temperature of, e.g. one hundred 50° F. To some extent, this will reduce the sitting time in which the dry components are allowed to remain in the oil or oil mixture.

Thereafter, the oil or oil mixture with the active ingredients of the dry components which have been dissolved by the oil or oil mixture is decanted from the remaining dry components. The oils could be filtered or strained from the dry components, if necessary. The dry components are thereafter discarded and the oil is preserved for use.

At this point in time, a preservative, such as a known preservative, benzoin, is then added to the oil. The oils can be heated to a temperature of about 150° F., as aforesaid. Moreover, heating is only necessary for a relatively short time as, for example, a period not normally exceeding about ten minutes. This heat allows the active components of the dry ingredients to be absorbed by the oil and to enable these other ingredients to also be absorbed by the oils.

Thereafter, the oil is stirred for several minutes. At this point, the oil is introduced into a wax or gel base which serves as the final carrier. The wax or gel base and the oils are then heated to enable the oils to be dissolved in the wax or gel base. Again, heating preferably should occur below a temperature of about 200° F. and then only for periods not exceeding five minutes.

After the heat is removed, the product is allowed to sit at room temperature to cool and thereby allow the wax or gel to harden.

It should be recognized that the entire percentage of the added dry ingredients are not dissolved in the oils and, hence, it is only the active ingredients which are removed by the oils. The precise exact ingredients and the percentages that are retained by the oils are not fully understood at this point in time. However, they have been found to be effective in the final composition.

When only the basic components, such as the willow, bergamot, alkanet, citronella oil and white willow are used, these components preferably exist by weight in ratios as set forth below. To the right of each component in the following Table I is set forth the allowed percentage range of each individual component. This is followed by the preferred percentage range by weight and, finally, this is followed by a specific percentage based on a most preferred embodiment of the invention.

TABLE I

| | Available | Preferred | Most Pre- |
|---|---|---|---|
| | Percentage | Percentage | ferred Embodi- |
| Ingredient | by Weight | by Weight | ment Percentage |
| Growth Stimulant | 6-17% | 8-14% | 11% |
| (bergamot) | | | |
| Microbicide | 6-17% | 8-14% | 11% |
| (citronella) | | | |
| Scalp Conditioner | 9-36% | 15-30% | 22% |
| (white willow) | | | |
| (bark) | | | |
| Hair Strengthener | 22-65% | 35-55% | 44% |
| (aklanet) | | | |
| Preservative | 6-17% | 8-14% | 11% |
| (benzoin) | | | |

TABLE II

| | Available | Preferred | Most Pre- |
|---|---|---|---|
| | Percentage | Percentage | ferred Embodi- |
| Ingredient | by Weight | by Weight | ment Percentage |
| Growth Stimulant | 5-16% | 7-13% | 10% |
| (bergamot) | | | |
| Microbicide | 5-15% | 7-13% | 10% |
| (citronella) | | | |
| Scalp Conditioner | 8-35% | 15-25% | 20% |
| (white willow) | | | |
| Hair strengthener | 20-60% | 30-50% | 40% |
| (alkanet) | | | |
| Preservative | 15-16% | 7-13% | 10% |
| (benzoin) | | | |
| Vitamin | 5-16% | 7-13% | 10% |
| (vitamin E) | | | |

When each of the aforesaid active components along with the two carrier oils and the wax or gel are used, they are also present in the weight range, as hereinafter described:

The important components of the composition preferably exist by weight in ratios as set forth below in Table III. To the right of each component in this following Table III is set forth the allowed percentage range of each individual component. Again, followed by the preferred percentage range by weight and, finally, by a specific percentage based on a most preferred embodiment of the invention:

TABLE III

| | Available | Preferred | Most Pre- |
|---|---|---|---|
| | Percentage | Percentage | ferred Embodi- |
| Ingredient | by Weight | by Weight | ment Percentage |
| Growth Stimulant | 0.05-0.24% | 0.08-0.17% | .128% |
| (bergamot) | | | |
| Microbicide | 0.05-0.24% | 0.08-0.17% | .128% |
| (citronella) | | | |
| Scalp Conditioner | 12-0.5% | 0.2-0.41% | .256% |
| (white willow) | | | |
| (bark) | | | |
| Hair strengthener | .2-1.5% | 0.37-0.71% | .512% |
| (alkanet) | | | |
| Preservative | 0.05-0.24% | 0.06-0.71% | .128% |
| (benzoin) | | | |
| Vitamin | 0.05-0.24% | 0.08-0.17% | .128% |
| (vitamin E) | | | |
| Carrier Oil | 60-95% | 71-89% | 82.72% |
| (soybean oil) | | | |
| Carrier Oil | 5-14% | 6-29% | 8% |
| (virgin olive oil) | | | |
| Hardener | 5-14% | 6-29% | 8% |
| (bees wax) | | | |

The oils which are used as the initial carrier are preferably soybean oil and olive oil. When used as a mixture, the soybean oil should be present in a predominant amount as, for example, about 90% by weight. However, the soybean oil can vary from about 60% to 100% by weight of the two oils. It is also possible to add other natural oils to the combination of soybean and olive oil, if desired, to act as a liquid carrier.

The waxes which are used as the final carrier or base for the composition are again preferably insect wax, such as beeswax, or a vegetable wax. It is also possible to use non-natural waxes or otherwise synthetic waxes, such as paraffin wax.

In order to form a gel composition, the gelling agent may be Irish moss or seaweed which are both natural gels. However, it is possible to use a synthetic gelling agent, such as carboxymethyl cellose or hydroxy methyl cellose.

### EXAMPLES

The invention is further illustrated by, but not limited to, the following example:

### Example 1

Approximately 83 ounces of soybean oil was mixed with approximately 8 ounces of olive oil to create an initial liquid carrier. The two oils were completely miscible with one another. Separately from the preparation of the oils, approximately 0.13 ounces of citronella was mixed with approximately 2.56 ounces of white willow and approximately 0.13 ounces of bergamot, as well as 13 ounces of vitamin E. In addition, approximately 0.512 ounces of alkanet was mixed with the other ingredients.

The mixture of active ingredients was then introduced into the mixture of soybean oil and olive oil and allowed to sit for approximately seventy-two hours at room temperature. Thereafter, the mixture of oils was decanted and preserved and the remaining dry ingredients were then discarded.

The oil mixture was then introduced into a vessel containing bees wax which was then heated causing a melting of the bees wax. The oils within a few minutes dissolved within the bees wax after heating to a temperature of about 200° F. The wax was then allowed to cool with the oils completely entrained therein.

In the preparation of the final composition with the wax base, approximately 8 ounces of wax was used to hold the oils and the other active ingredients.

Testing of the composition found that the composition did tend to stimulate hair growth and did remove incidents of dandruff. It was also found that the composition freed the scalp from any microbial growth, such as fungal growth, for a substantial period of time.

Thus, there has been illustrated and described a unique and novel hair and scalp treatment composition and which thereby fulfills all of the objects and advantages which have been sought. It should be understood that many changes, modifications, variations and other uses and applications which will become apparent to those skilled in the art after considering the specification and the accompanying drawings. Therefore, any and all such changes, modifications, variations and other uses and applications which do not depart from the spirit and scope of the invention are deemed to be covered by the invention.

Having thus described the invention, what we desire to claim and secure by Letters Patent is:

1. A hair and scalp treatment composition primarily formulated of natural ingredients and capable of providing hair and scalp treatments without any substantial damage to the hair or scalp, said composition comprising:
a) a natural hair growth stimulant;
b) a natural microbicidal acting agent;
c) a natural scalp conditioner;
d) a natural hair straightening agent;
e) a preservative which is compatible with and operates to extend shelf life of the hair growth stimulant, microbicidal acting agent and scalp conditioner and hair straightener; and
f) a natural carrier oil for receiving active ingredients from the components in (a) through (d) above.

2. The hair and scalp treatment composition of claim 1 further characterized in that:
a) the hair growth stimulant is bergamot;
b) the natural microbicidal acting agent is citronella oil;
c) the natural scalp conditioner is a willow bark; and
d) the hair straightening agent is alkanet.

3. The hair and scalp treatment composition of claim 2 further characterized in that the preservative is benzoin.

4. The hair and scalp treatment composition of claim 1 further characterized in that the composition also includes a vitamin therein.

5. The hair and scalp treatment composition of claim 3 further characterized in that said composition comprises vitamin E.

6. The hair and scalp treatment composition of claim 5 further characterized in that the aforesaid hair growth stimulant, natural microbicidal acting agent, the natural scalp conditioner, the natural hair straightening agent, and the vitamin E are carried by soybean oil.

7. The hair and scalp treatment composition of claim 6 further characterized in that a pair of carrier oils are used and comprise soybean oil and virgin olive oil.

8. The hair and scalp treatment composition of claim 4 further characterized in that the carrier oils and the other ingredients of the composition are carried in a hardener to make a wax composition.

9. The hair and scalp treatment composition of claim 8 further characterized in that the hardener is bees wax.

10. The hair and scalp treatment composition of claim 1 further characterized in that:
a) the hair growth stimulant is present in an amount of about 0.05 to 0.024% by weight based on the total weight of the composition;
b) the microbicide is present in an amount of about 0.05% by weight to about 0.024% by weight based on the total weight of the composition;
c) the scalp conditioner in present in an amount of about 0.12% to about 0.5% by weight based on the total weight of the composition;
d) the hair straightening agent is present in an amount of about 0.2% to about 1.5% by weight based on the total weight of the composition; and
e) the preservation is present in an amount of about 0.05% to about 0.24% by weight.

11. The hair and scalp treatment composition of claim 1 further characterized in that:
a) the hair growth stimulant is present in an amount of about 0.08 to 0.17% by weight based on the total weight of the composition;
b) the microbicide is present in an amount of about 0.08% by weight to about 0.17% by weight based on the total weight of the composition;
c) the scalp conditioner in present in an amount of about 0.2% to about 0.41% by weight based on the total weight of the composition;
d) the hair straightening agent is present in an amount of about 0.37% to about 0.71% by weight based on the total weight of the composition; and
e) the preservation is present in an amount of about 0.08% to about 0.17% by weight.

12. The hair and scalp treatment composition of claim 11 further characterized in that a vitamin is present in an amount of about 0.05% to about 0.24% by weight.

13. The hair and scalp treatment composition of claim 12 further characterized in that the vitamin is vitamin E and is present in an amount of about 0.08% to about 0.17% by weight.

14. The hair and scalp treatment composition of claim 10 further characterized in that each of the aforesaid active ingredients are carried in a carrier oil which is present in an amount of about 60% to about 95% by weight.

15. The hair and scalp treatment composition of claim 14 further characterized in that the carrier oil is present is an amount of about 71% to about 89% by weight based on the total weight of the composition.

16. The hair and scalp treatment composition of claim 15 further characterized in that the carrier oil is soybean oil.

17. The hair and scalp treatment composition of claim 11 further characterized in that:
a) the hair growth stimulant is present in an amount of about 0.12% by weight;
b) the microbicide is present in an amount of about 0.12% by weight;
c) the scalp conditioner is present in an amount of about 0.256% by weight;
d) the hair strengthening agent is present in an amount of about 0.512% by weight; and
e) the preservative is present in an amount of about 0.128% by weight.

18. The hair and scalp treatment composition of claim 17 further characterized in that it contains a vitamin present in an amount of about 0.128% by weight.

19. The hair and scalp treatment composition of claim 11 further characterized in that growth stimulant, microbicide, scalp conditioner, hair straightening agent, preservative and vitamin are carried in an initial carrier oil present in an amount about 82% to 83% by weight and which is then dissolved in a wax.

20. A method for treatment of the hair and scalp of an individual to stimulate the growth of hair and to preclude microbicidal acting agents from acting on the scalp and hair and also conditioning and straightening the hair with natural ingredients, said method comprising:
a) applying to the scalp and hair of an individual a composition containing:
1) a natural hair growth stimulant;
2) a natural microbicidal acting agent;
3) a natural scalp conditioner;
4) a natural hair straightening agent;
5) a preservative which is compatible with and operates to external shelf life of the hair growth stimulant, microbicidal acting agent and scalp conditioner and hair straightener; and
6) a natural carrier oil for receiving active ingredients from the components in (a) through (d) above.

21. The method of treating the hair and scalp of claim 20 further characterized in that the method comprises applying to the hair a composition as set forth in claim 20 and further comprising a vitamin therein.

22. The method of treating the hair and scalp of claim 20 further characterized in that the method comprises applying to the hair a composition as set forth in claim 20 and further comprising vitamin E therein.

23. The method of treating the hair and scalp of claim 20 further characterized in that the method comprises applying to the hair a composition as set forth in claim 20 and wherein the components thereof are carried in a carrier oil.

24. The method of treating the hair and scalp of claim 20 further characterized in that the method comprises applying to the hair a composition as set forth in claim 20 and wherein the carrier oil is soybean oil.

25. The method of treating the hair and scalp of claim 20 further characterized in that the method comprises applying to the hair a composition as set forth in claim 20 and wherein the carrier oil is a combination of soybean oil and virgin olive oil.

26. The method of treating the hair and scalp of claim 25 further characterized in that the method comprises applying to the hair a composition as set forth in claim 20 and wherein the carrier oils and the other ingredients of the composition are carried in a hardener to make a wax composition.

27. The method of treating the hair and scalp of claim 25 further characterized in that the method comprises applying to the hair a composition as set forth in claim 20 and wherein the wax is bees wax.

28. The method of treating the hair and scalp of claim 20 further characterized in that the method comprises applying to the hair a composition as set forth in claim 20 and further comprising:
a) the hair growth stimulant is present in an amount of about 0.05 to 0.024% by weight based on the total weight of the composition;
b) the microbicide is present in an amount of about 0.05% by weight to about 0.024% by weight based on the total weight of the composition;
c) the scalp conditioner in present in an amount of about 0.12% to about 0.5% by weight based on the total weight of the composition;
d) the hair straightening agent is present in an amount of about 0.2% to about 1.5% by weight based on the total weight of the composition; and
e) the preservation is present in an amount of about 0.05% to about 0.24% by weight.

29. The method of treating the hair and scalp of claim 20 further characterized in that the method comprises applying to the hair a composition as set forth in claim 20 and further comprising:
a) the hair growth stimulant is present in an amount of about 0.08 to 0.17% by weight based on the total weight of the composition;
b) the microbicide is present in an amount of about 0.08% by weight to about 0.17% by weight based on the total weight of the composition;
c) the scalp conditioner in present in an amount of about 0.2% to about 0.41% by weight based on the total weight of the composition;
d) the hair straightening agent is present in an amount of about 0.37% to about 0.71% by weight based on the total weight of the composition; and
e) the preservation is present in an amount of about 0.08% to about 0.17% by weight.

30. The method of treating the hair and scalp of claim 29 further characterized in that the method comprises applying to the hair a composition as set forth in claim 29 and further comprising a vitamin is present in an amount of about 0.05% to about 0.24% by weight.

31. The method of treating the hair and scalp of claim 30 further characterized in that the method comprises applying to the hair a composition as set forth in claim 20 and further comprising the vitamin is E and is present in an amount of about 0.08% to about 0.17% by weight.

32. The method of treating the hair and scalp of claim 20 further characterized in that the method comprises applying to the hair a composition as set forth in claim 20 and further comprising the active ingredients are carried in a carrier oil which is present in an amount of about 60% to about 95% by weight.

33. The method of treating the hair and scalp of claim 20 further characterized in that the method comprises applying to the hair a composition as set forth in claim 20 and further comprising:
a) the hair growth stimulant is present in an amount of about 0.12% by weight;
b) the microbicide is present in an amount of about 0.12% by weight;
c) the scalp conditioner is present in an amount of about 0.256% by weight;
d) the hair strengthening agent is present in an amount of about 0.512% by weight; and
e) the preservative is present in an amount of about 0.128% by weight.

34. The method of treating the hair and scalp of claim 20 further characterized in that the method comprises applying to the hair a composition as set forth in claim 20 and further comprising a vitamin present in an amount of about 0.128% by weight.
